(19) United States
(12) Patent Application Publication
(10) Pub. No.: US 2026/0105406 A1
(43) Pub. Date: Apr. 16, 2026

(54) SYSTEMS AND METHODS FOR AGGREGATING SUPPLY ORDERS FOR PHARMACEUTICAL TRIALS

(71) Applicant: R.P. Scherer Technologies, LLC, Carson City, NV (US)

(72) Inventors: Mark MCCORRY, South Lanarkshire (GB); Sakunthaladevi IYADURAI, Lawrenceville, NJ (US); Stephen FLAHERTY, Greater Manchester (GB); Andrew FREEMAN, Lancashire (GB)

(73) Assignee: R.P. Scherer Technologies, LLC, Carson City, NV (US)

(21) Appl. No.: 19/116,661

(22) PCT Filed: Sep. 25, 2023
(86) PCT No.: PCT/US2023/075025

§ 371 Date: Mar. 28, 2025

Related U.S. Application Data
(60) Provisional application No. 63/411,333, filed on Sep. 29, 2022.

(51) Int. Cl. G06Q 10/083; G16H 10/20

(52) U.S. Cl. CPC G06Q 10/083; G16H 10/20

(57) ABSTRACT

Embodiments of the present disclosure include systems and methods for confidentially producing shipment orders for clinical trials. The method includes receiving a plurality of supply orders associated with one or more clinical trials and determining a clinical trial study, a site location, and a treatment type. A first plurality of supply orders are assigned to a packaging site based on the site location. The method further includes, associating a first sub-group of the first plurality of supply orders with a first assembly procedure, associating a second sub-group of the plurality of supply orders with a second assembly procedure, and assigning, randomly, the plurality of kit identifiers to the first sub-group and the second subgroup, thereby creating a plurality of blinded kits. The method further includes confidentially producing shipment orders for the first plurality of clinical trials based on the plurality of blinded kits.

### CROSS-REFERENCE TO RELATED APPLICATIONS

This application claims the benefit of U.S. Provisional Application No. 63/411,333 filed Sep. 29, 2022, the entire contents of which are incorporated herein by reference.

### FIELD

The present disclosure relates generally to systems and methods for producing shipment orders for clinical trial studies, and more specifically, for producing confidential shipment orders for blinded clinical trial studies.

### BACKGROUND

Companies engaging in clinical trial studies, particularly, pharmaceutical clinical trials are required to adhere to government regulated clinical packaging and labeling standards. For example, various national bodies like the Food and Drug Administration (FDA), Medicines and Healthcare products Regulatory Agency (MHRA) and European Medicines Agency (EMA) provide a regulatory framework for clinical packaging. As such, each clinical packaging job, whether a primary (e.g., putting bulk drug into bottle, blister, vial or similar) or a secondary packaging job (e.g., clinically labeling a primary bottle and putting it into a box and labeling the box to create a patient kit), must be controllable and repeatable in action and follow appropriate regulatory guidelines. Moreover, the packaging should be performed in a way that does not reveal which kits are associated with which treatment types (e.g., unblind the clinical trial by allowing a participant or another individual involved with the clinical trial to discover whether a kit is an active treatment or a placebo).

Traditionally, packaging methods for clinical trial studies may be performed to prepare a large quantity of kits including the medication for the clinical trial study. The kits may include a primary packaging vessel (e.g., bottle, blister pack, vial, or the like that holds the medication) and a secondary packaging vessel (e.g., box that contains the primary packaging vessel and instructions). These kits may be stored in a facility and packaged for shipment upon receiving a request for a quantity of kits to be sent to a specific clinical trial site location. More recently, there has been a shift from pre-packaging and storing large quantities of kits to providing just in time packaging, where the kits are packaged at or around the time the request is received. The just in time packaging may reduce waste by packaging what is requested. For example, waste can be reduced when there is a limited supply of a drug, the clinical trial study does not progress in a particular country, or the clinical trial study does not receive an expected level of patient participation.

There can be inefficiencies associated with just in time packaging. Clinical trial studies often include one or more active medication or treatment types and a control or placebo treatment type. During the packaging process, each active treatment type and the control treatment type are prepared and packaged in separate packaging runs. Each packaging run may be associated with a packaging room set-up, a packaging room clean-down, and a separate review and release of packaged product for each treatment type. For just in time packaging, where the quantities of kits are relatively small, the time and cost may be substantial. Additionally, the packaging should be performed in a way that does not reveal which kits are associated with which treatment types (e.g., unblind the study by allowing a participant or another individual involved with the study to discover whether a kit is an active treatment or placebo). Accordingly, there is a need to confidentially and efficiently package kits for clinical trial studies.

### SUMMARY

Embodiments of the present disclosure can provide computational systems and methods for efficiently aggregating supply orders. Each supply order may be associated with a destination site associated with a site location that may distribute one or more products or kits to participants in the clinical trial study. In one or more examples, the aggregation of multiple supply orders may be performed to create one or more larger assembly orders, where each assembly order is associated with a particular treatment type (e.g., an active treatment type or a control treatment type). Each assembly order may correspond to a packaging run to prepare kits for one or more clinical trial site locations. Embodiments of the present disclosure may further dis-aggregate the kits produced in the packaging runs to produce shipment orders based on the original supply orders.

In one or more examples, embodiments of the present disclosure can improve efficiency to just in time packaging processes, where the clients may be placing supply orders for immediate need at site location in real time. As a result, each supply order may correspond to a relatively small packaging run (e.g., including less than 100 kits) and not to long term demand (greater than 100 kits). In some examples, the supply order may include quantities of less than five kits. Because each packaging run may be associated with a room set-up, a room clean-down, and a separate review and release of the packaged product, packaging supply orders to meet real time demand may be time consuming and costly. Aggregating supply orders according to embodiments of the present disclosure may lead to improved efficiencies by reducing the time and cost associated with multiple supply runs for a single supply order.

Moreover, embodiments of the present disclosure may lead to improved confidentiality in producing shipment orders for blinded studies. In many instances, in order for a clinical trial to be successful, both the participants and individuals running the clinical trial should not know which medication kits correspond to an active treatment type or a control treatment type. Oftentimes, a randomization list is used to map the kits (e.g., kit identifiers) to a treatment type (e.g., active or control). Embodiments of the present disclosure provide improved methods of maintaining the confidentiality of the randomization list by restricting access to the randomization list. In one or more examples, this is achieved by maintaining the randomization list on a computer system associated with a packaging facility (e.g., the facility that packages the kits). In one or more examples, other portions of the system may not have access to the randomization list.

Embodiments of the present disclosure provide methods for confidentially producing shipment orders for clinical trials. In one or more examples, the method comprises at a first computer system comprising one or more processors and one or more memories: receiving a plurality of supply orders associated with one or more clinical trials, determining for each of the plurality of supply orders: a clinical trial study, a site location, and a treatment type, assigning a first plurality of supply orders associated with a first clinical trial study to a packaging site based on the site location. In one or more examples, the method further comprises associating a first sub-group of the first plurality of supply orders with a first assembly procedure based on the treatment type to create a first assembly order, and associating a second sub-group of the first plurality of supply orders with a second assembly procedure based on the treatment type to create a second assembly order. In one or more examples, the method further comprises at a second computer system comprising a second one or more processors and a second one or more memories: receiving the first assembly order and the second assembly order; and assigning, randomly, a plurality of kit identifiers to the first assembly order and the second assembly order, thereby creating a plurality of blinded kits. In one or more examples, the method further comprises at the first computer system, confidentially producing shipment orders for the first plurality of clinical trials based on the plurality of blinded kits.

In one or more examples, embodiments of the present disclosure include an electronic system comprising one or more processors, a memory, and one or more programs, wherein the one or more programs are stored in the memory and configured to be executed by the one or more processors. In one or more examples, the one or more programs further include instructions for at a first computer system comprising one or more processors and one or more memories: receiving a plurality of supply orders associated with one or more clinical trials, determining for each of the plurality of supply orders: a clinical trial study, a site location, and a treatment type, assigning a first plurality of supply orders associated with a first clinical trial study to a packaging site based on the site location. In one or more examples, the one or more programs further include instructions for associating a first sub-group of the first plurality of supply orders with a first assembly procedure based on the treatment type to create a first assembly order, and associating a second sub-group of the first plurality of supply orders with a second assembly procedure based on the treatment type to create a second assembly order. In one or more examples, the one or more programs include instructions for at a second computer system comprising a second one or more processors and a second one or more memories: receiving the first assembly order and the second assembly order, and assigning, randomly, a plurality of kit identifiers to the first assembly order and the second assembly order, thereby creating a plurality of blinded kits. In one or more examples, the one or more programs include instructions for at the first computer system, confidentially producing shipment orders for the first plurality of clinical trials based on the plurality of blinded kits.

In one or more examples, embodiments of the present disclosure further include a non-transitory computer-readable storage medium storing one or more programs, the one or more programs comprising instructions, which when executed by one or more processors of one or more electronic devices having a display, cause the one or more electronic devices to: at a first computer system comprising one or more processors and one or more memories, receive a plurality of supply orders associated with one or more clinical trials, determine for each of the plurality of supply orders: a clinical trial study, a site location, and a treatment type, assign a first plurality of supply orders associated with a first clinical trial study to a packaging site based on the site location. In one or more examples, the one or more electronic devices are further caused to: associate a first sub-group of the first plurality of supply orders with a first assembly procedure based on the treatment type to create a first assembly order, and associate a second sub-group of the first plurality of supply orders with a second assembly procedure based on the treatment type to create a second assembly order. In one or more examples, the one or more electronic devices are further caused to: at a second computer system comprising a second one or more processors and a second one or more memories, receive the first assembly order and the second assembly order, and assign, randomly, a plurality of kit identifiers to the first assembly order and the second assembly order, thereby creating a plurality of blinded kits. In one or more examples, the one or more electronic devices are further caused to at the first computer system, confidentially produce shipment orders for the first plurality of clinical trials based on the plurality of blinded kits.

### BRIEF DESCRIPTION OF THE FIGURES

FIG. 1 illustrates an exemplary process for confidentially producing shipment orders, in accordance with some embodiments of this disclosure.

FIG. 2 illustrates an exemplary assembly order, in accordance with some embodiments of this disclosure.

FIGS. 3A-3E illustrate exemplary processes for confidentially producing shipment orders, in accordance with some embodiments of this disclosure.

FIGS. 4A-4J illustrate exemplary user interfaces for confidentially producing shipment orders, in accordance with some embodiments of this disclosure.

FIG. 5 illustrates an exemplary electronic device, in accordance with embodiments of this disclosure.

### DETAILED DESCRIPTION

Embodiments of the present disclosure can provide systems and methods for efficiently aggregating supply orders. Each supply order may be associated with a destination site associated with a site location that may distribute one or more products or kits to participants in the clinical trial study. In one or more examples, the aggregation of supply orders may be performed to create one or more assembly orders, where each assembly order is associated with a particular treatment type (e.g., an active treatment type or a control treatment type). Each assembly order may correspond to a packaging run to prepare kits for one or more clinical trial site locations. Embodiments of the present disclosure may further dis-aggregate the kits produced in the packaging runs to produce shipment orders based on the supply orders.

In one or more examples, embodiments of the present disclosure can improve efficiency in just in time packaging processes, where the clients may be placing supply orders for immediate need at site locations in real time. In just in time packaging processes, each supply order may correspond to a relatively small packaging run (e.g., including less than 100 kits). In some examples, the supply order may include quantities of less than five kits. Because each packaging run may be associated with a room set-up, a room clean-down, and a separate review and release of the packaged product, packaging supply orders to meet real time demand may be time consuming and costly. Aggregating supply orders according to embodiments of the present disclosure may lead to improved efficiencies by reducing the time and cost associated with multiple supply runs for a single supply order.

Moreover, embodiments of the present disclosure may lead to improved confidentiality in producing shipment orders for blinded studies. In many instances, in order for a clinical trial to be successful, both the participants and individuals running the clinical trial should not know which medication kits correspond to an active treatment type or a control treatment type. Oftentimes, a randomization list is used to map the kits (e.g., kit identifiers) to a treatment type (e.g., active or control), whether the randomization is performed by the entity running the clinical trial or a vendor or service provider associated with the entity. Embodiments of the present disclosure provide improved methods of maintaining the confidentiality of the randomization list by restricting access to the randomization list. In one or more examples, this is achieved by maintaining the randomization list on a computer system associated with a packaging facility (e.g., the facility that packages the kits). In one or more examples, other portions of the system may not have access to the randomization list.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Although the following description uses terms “first,” “second,” etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

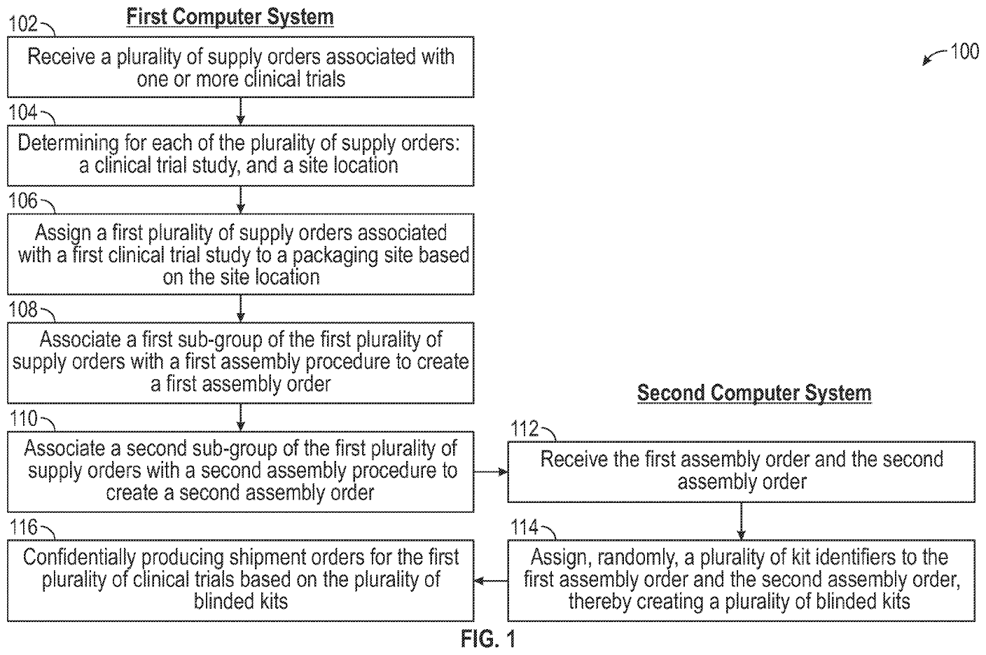

FIG. 1 illustrates an exemplary process 100 for confidentially producing shipment orders for one or more clinical trial studies, the shipment orders comprising one or more blinded kits. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At block 102 of FIG. 1, the system can, at a first computer system, receive a plurality of supply orders associated with one or more clinical trials. The plurality of supply orders can be related to a particular clinical trial study associated with a client.

In one or more examples, the system can receive the plurality of supply orders via a direct communication, e.g., email, telephone call, letter, etc., from the client. In one or more examples, the system can receive the plurality of supply orders via an external system or application. In one or more examples, the external system may be integrated with the system via an application programming interface (API). For example, the external system may be configured to communicate with the system upon receiving the plurality of supply orders via the API.

At block 104 of FIG. 1, the system can, at the first computer system, determine for the plurality of supply orders: a clinical trial study, and a site location. In one or more examples, the system can determine additional information associated with the plurality of supply orders.

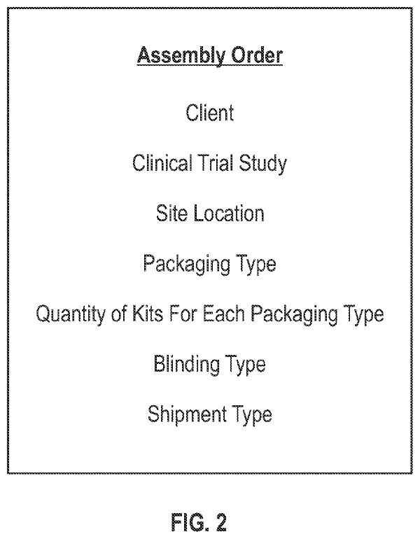

FIG. 2, illustrates exemplary data that may be associated with a supply order. As shown in the figure, a supply order may include client data, clinical trial study data, site location data, a packaging type, a quantity for each packaging type, a blinding type, and a shipment type. The type of information shown in FIG. 2 is exemplary and more or less information may be associated with a supply order without departing from the scope of this disclosure.

In one or more examples, the client data may indicate the identity of the client that requested the supply order. In one or more examples, the clinical trial study may associate the supply order with a particular clinical trial study. In one or more examples, the clinical trial study may be identified by a number corresponding to a particular clinical trial study. In one or more examples, the site location can correspond to a particular location associated with the clinical trial study that should receive the product requested via the supply order. In one or more examples, the packaging type may indicate the type of material (e.g., medication, dosage for drugs, bottles, syringes, labels, etc.). For example, a first packaging type associated with a clinical study may correspond to a first dosage for a specific medication to be provided in a bottle with a first type of label, a second packaging type associated with the same clinical study may correspond to a placebo to be provided in the bottle with the first type of label, and a third packaging type associated with the clinical study may correspond to a second dosage for the specific medication to be provided in the bottle with the first type of label. A skilled artisan will understand that the number of packaging types, medications and dosages prescribed will vary based on the parameters of the clinical trial study. In one or more examples, the quantity of kits for each packaging type may indicate how many kits for each packaging type have been ordered. In one or more examples, the blinding type may indicate whether the study is blinded. In one or more examples, the shipment type can refer to a pack to order shipment or a pack to stock shipment.

As an example, a client may be planning a clinical trial to test a particular medication and evaluate the efficacy and side effects associated with the medication. In such examples, the system may receive a plurality of supply orders associated with the same clinical trial. The plurality of supply orders may be associated with multiple clinical trial site locations where the medication will be distributed to participants of the clinical trial study. In one or more examples, each clinical trial site location may be associated with a different number of kits. For example, a clinical trial study may be associated with locations in Philadelphia, PA and Urbana, IL. The Philadelphia location may be expected to include a higher number of participants, accordingly, the quantity of kits for these locations may differ.

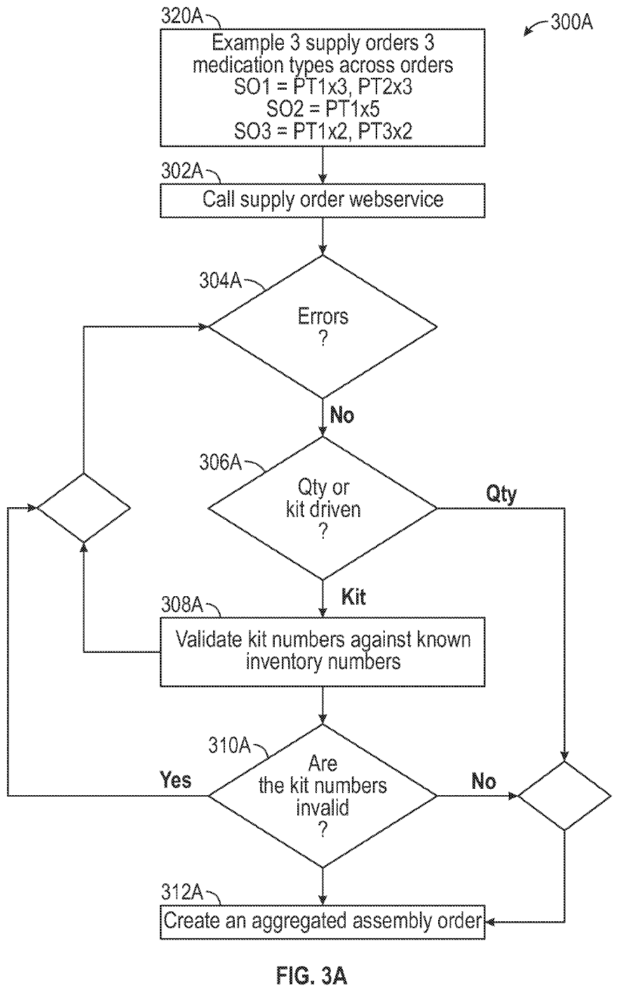

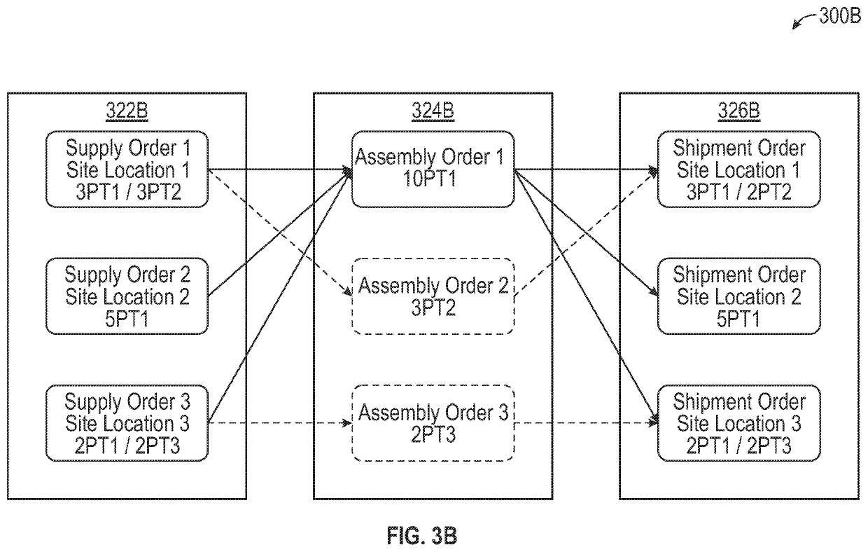

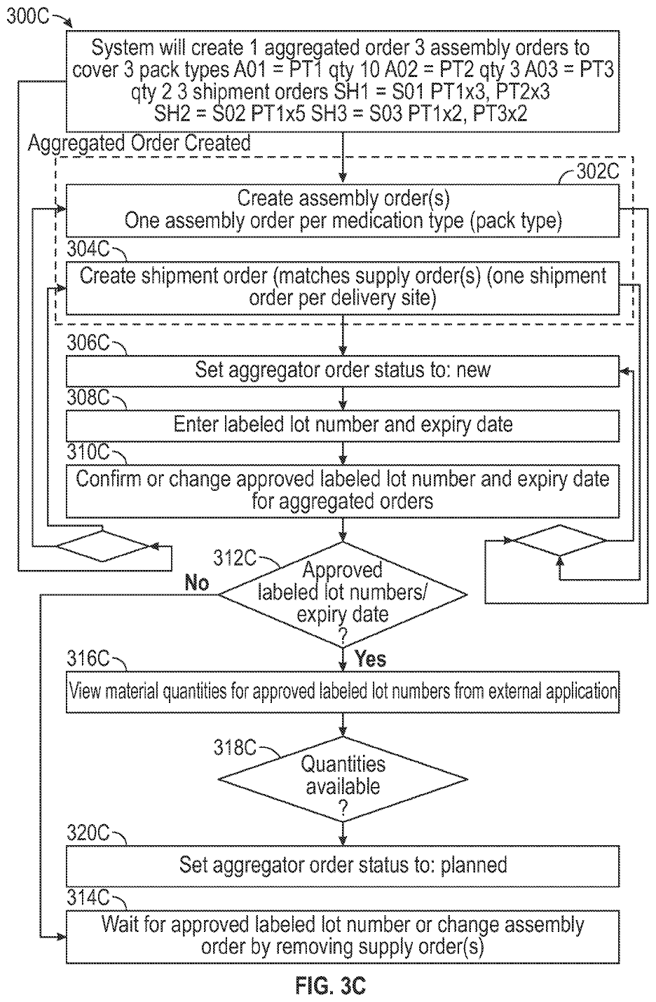

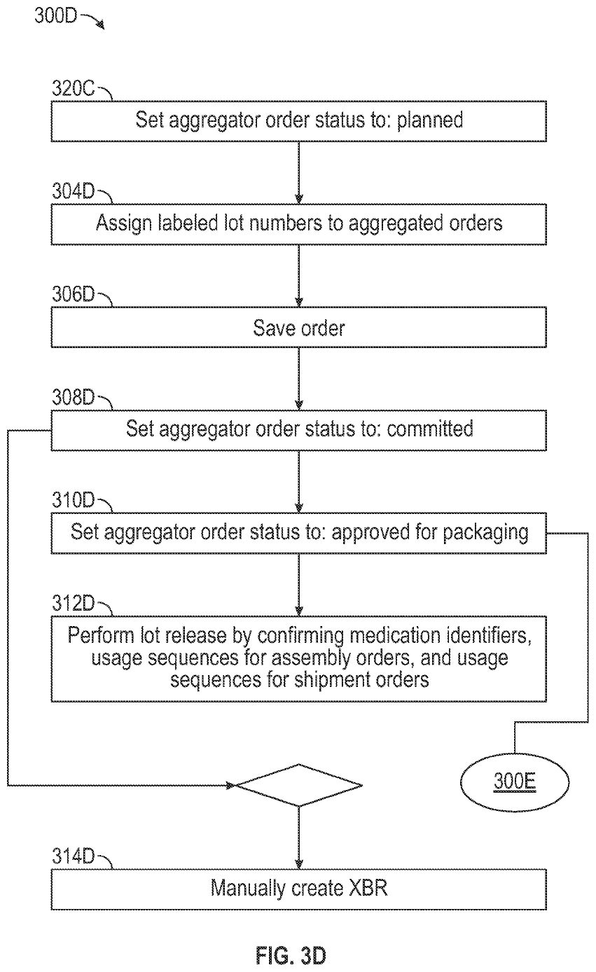

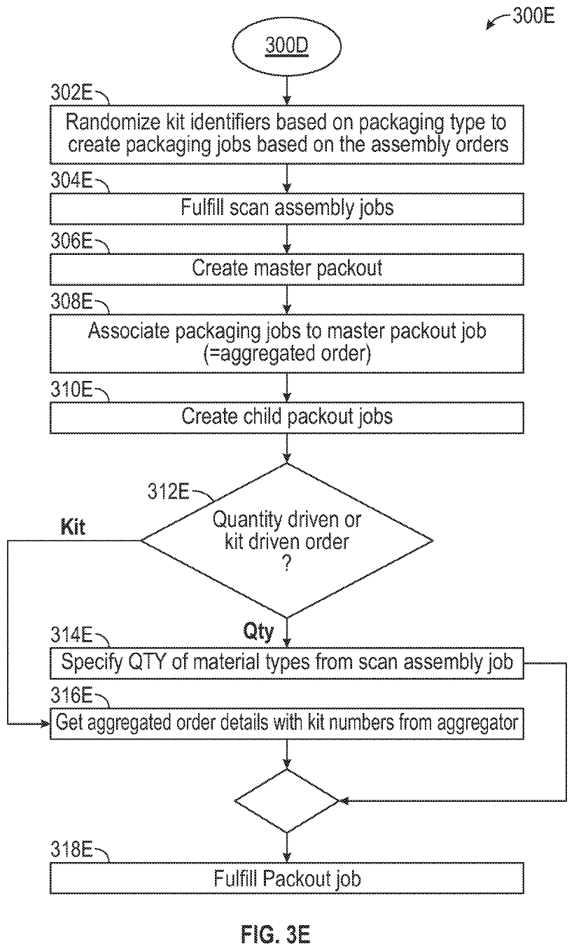

FIG. 3A, illustrates an exemplary process 300A associated with blocks 102 and 104. At block 302, the system can receive supply orders 320A, by for example, calling a supply order webservice. In one or more examples, the supply order webservice can include an API associated with an external system. As shown in the figure, supply orders 320A may include three supply orders that may be associated with three medication types across the orders. For example, the first supply order may be associated with three products of a first packaging type (SO1=PT1x3) and three products of a second packaging type (SO1=PT2x3); the second supply order may be associated with five products of the first packaging type (SO2=PT1x5); and the third supply order may be associated with two products of the first packaging type (SO3=PT1x2) and two products of a second packaging type (SO3=PT3x2). As used herein, the products may refer to a packaged kit intended to be distributed to a clinical trial study participant. The products or kits may include a predetermined quantity of a specific medication packaged in a predetermined vessel (e.g., bottle, blister packs, syringes, etc.) with predetermined labels. In one or more examples, the first packaging type may correspond to a first medication type at a first dosage, the second packaging type may correspond to a placebo, and the third packaging type may correspond to the first medication type at a second dosage. For example, the first packaging type may correspond to a dose of 100 mg of aspirin, the second packaging type may correspond to a placebo, and the third packaging type may correspond to a dose of 50 mg of aspirin. A skilled artisan will understand that these medications and dosages are exemplary and any medications and dosages may be used without departing from the scope of this disclosure.

At Block 304A, the system may check for errors associated with the received supply orders 320A. If errors are detected, the system may communicate with the external system to resolve the error. If no errors are detected, the system may determine if the received supply orders 320A are quantity driven or kit driven, at block 306A. Each of the kits may be associated with a unique serial identification number. In one or more examples, a quantity driven order may refer to a supply order where the client specifies a specific number of kits and is agnostic to the unique serial identification numbers associated with the kits. In one or more examples, a kit driven supply order may refer to a supply order where the client specifies specific serial identification numbers. If the system determines that the supply orders are kit driven, the system may proceed to block 308A, where the system can validate kit numbers against known inventory numbers. The system can determine whether the kit numbers are invalid at block 310A. If the numbers are invalid, the system may flag an error. If the kit numbers can be validated against known inventory numbers the system may create an aggregated supply order at block 312A. If the system determines that the supply orders are quantity driven, then the system may proceed to create an aggregated supply order at block 312A.

Returning to FIG. 1, at block 106, the system can, at the first computer system, assign a first plurality of supply orders associated with a first clinical trial study to a packaging site based on the site location. In one or more examples, the first plurality of supply orders can correspond to supply orders associated with a plurality of site locations within the same country. For examples, different countries may have different rules and regulations regarding providing and packaging medication for use in clinical trials. Accordingly, a first plurality of supply orders for site locations in the same country will be subject to the same packaging requirements. For example, if the system receives a plurality of supply orders with clinical trial study site locations in the US, Canada, and Japan. In such an example, the first plurality of supply orders may be associated with clinical trial studies located in the US; a second plurality of supply orders may be associated with clinical trial studies located in Canada; and a third plurality of supply orders may be associated with clinical trial studies located in Japan. In one or more examples, the plurality of supply orders may be associated with one or more countries, as permitted by the design of the clinical trial study.

In one or more examples, the system may automatically assign a packaging site to the first plurality of supply orders based on the clinical trial site location. In one or more examples, the packaging site may be based pre-determined based on the country where the site locations are located. In one or more examples, the packaging site may be based on whether the site locations are located in the same country. In one or more examples, the packaging site may be based on whether the site locations are located in the same region. In one or more examples, the packaging location may be pre-configured such that when a request from a particular site location is received, the packaging site may be automatically assigned.

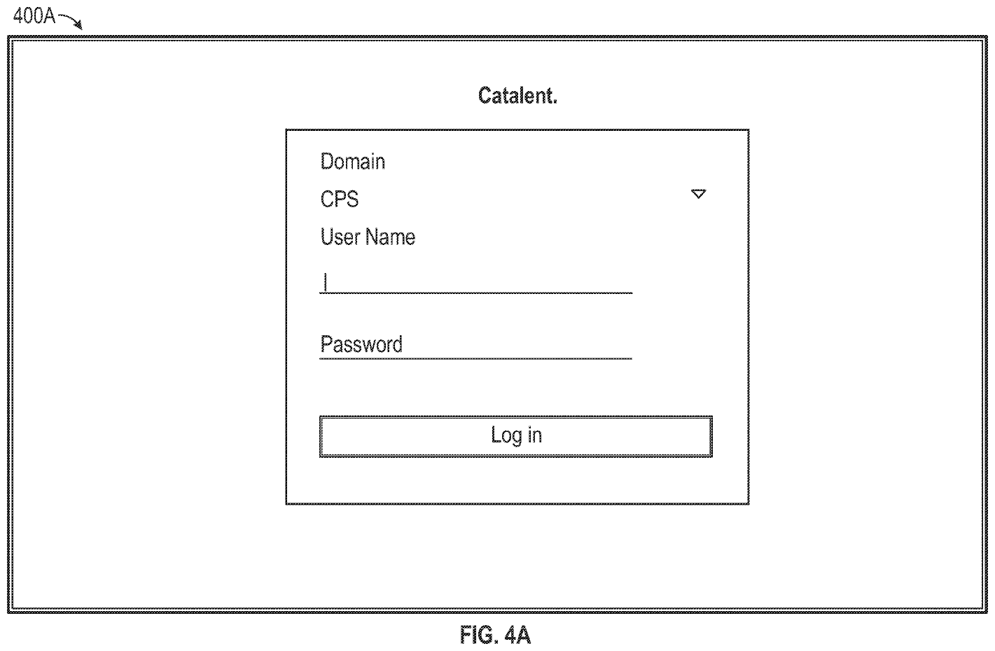

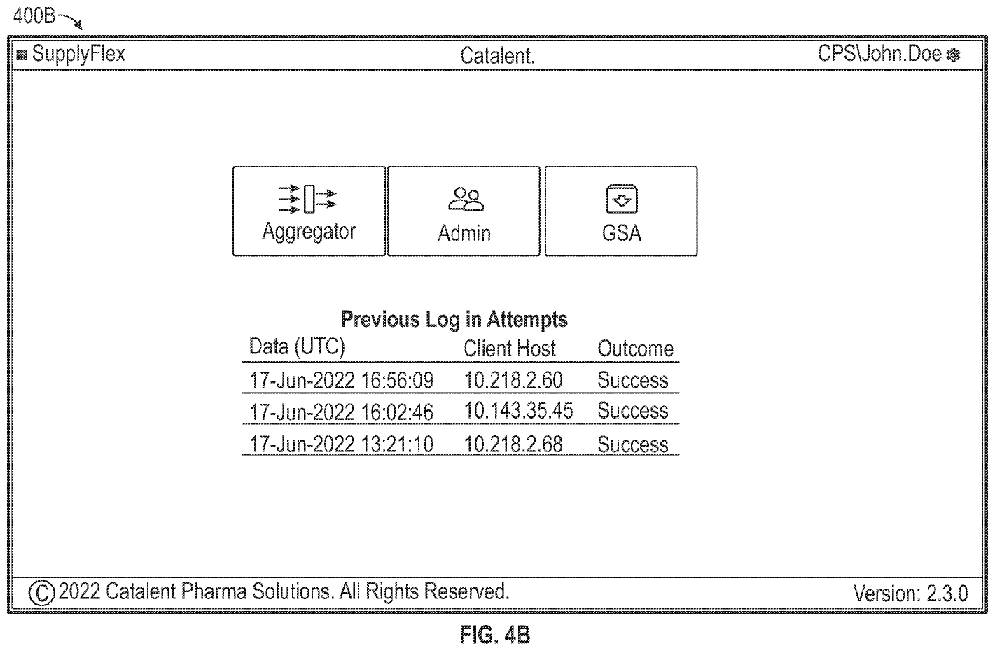

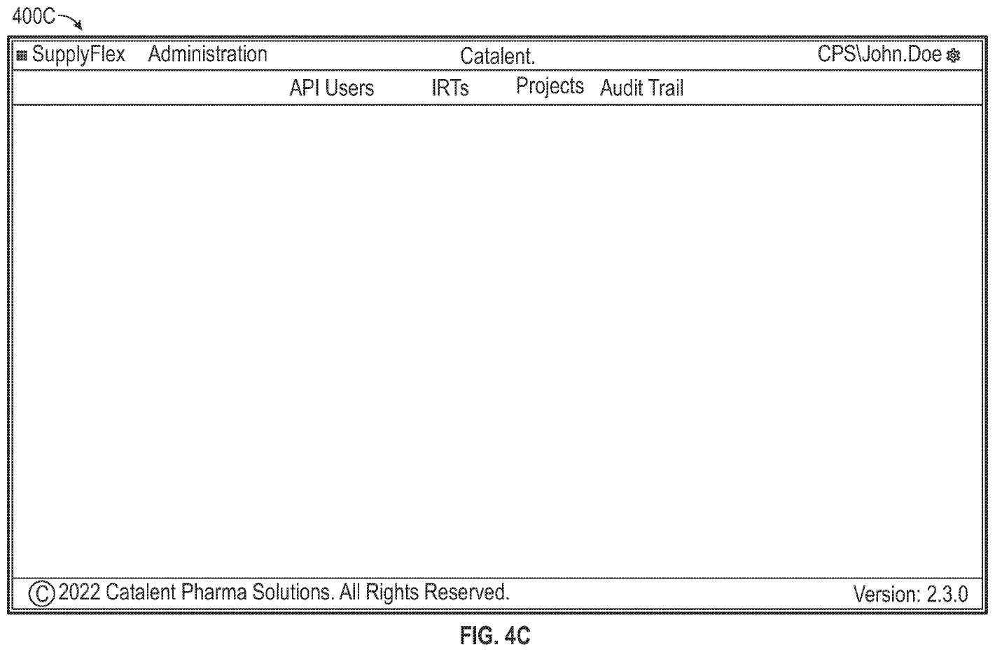

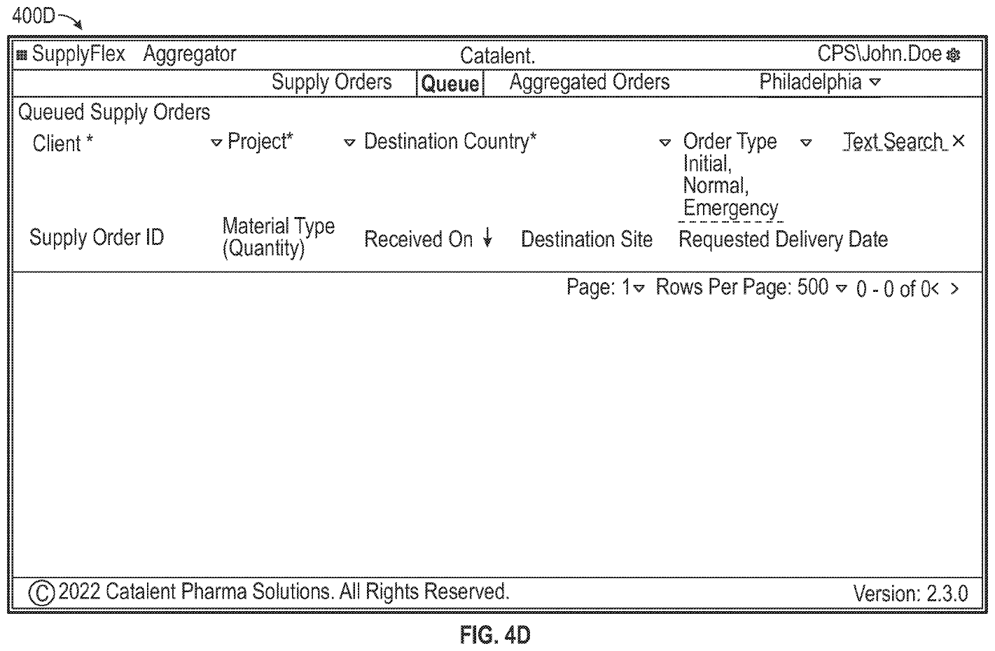

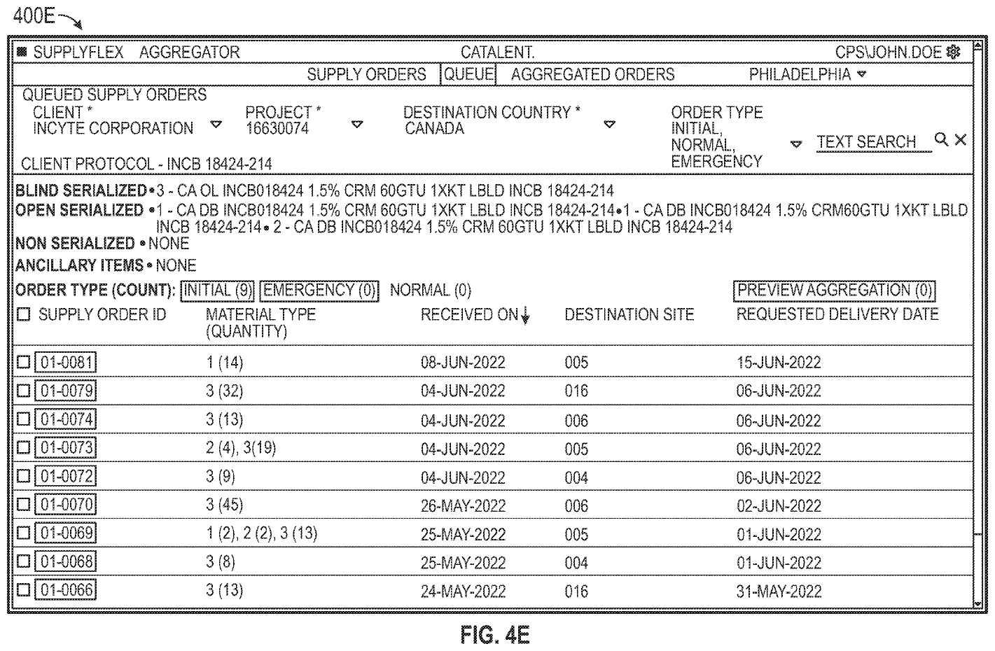

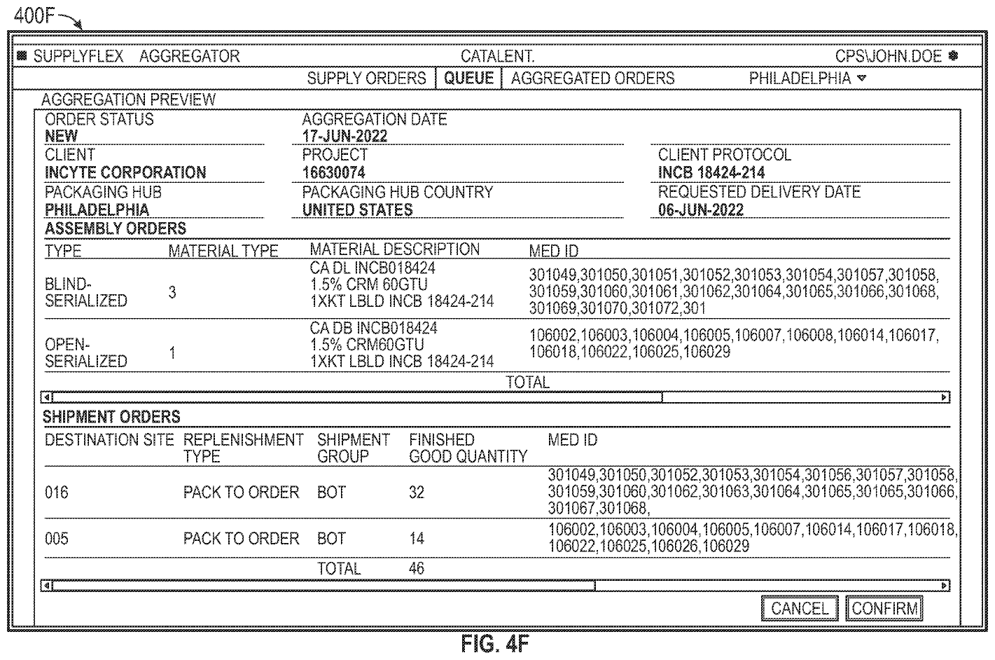

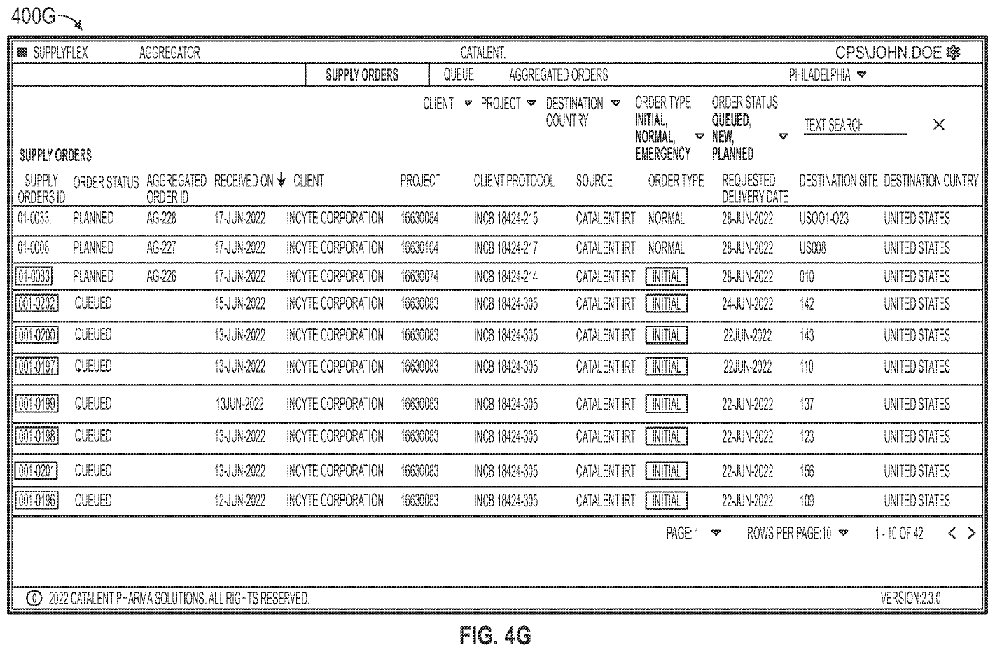

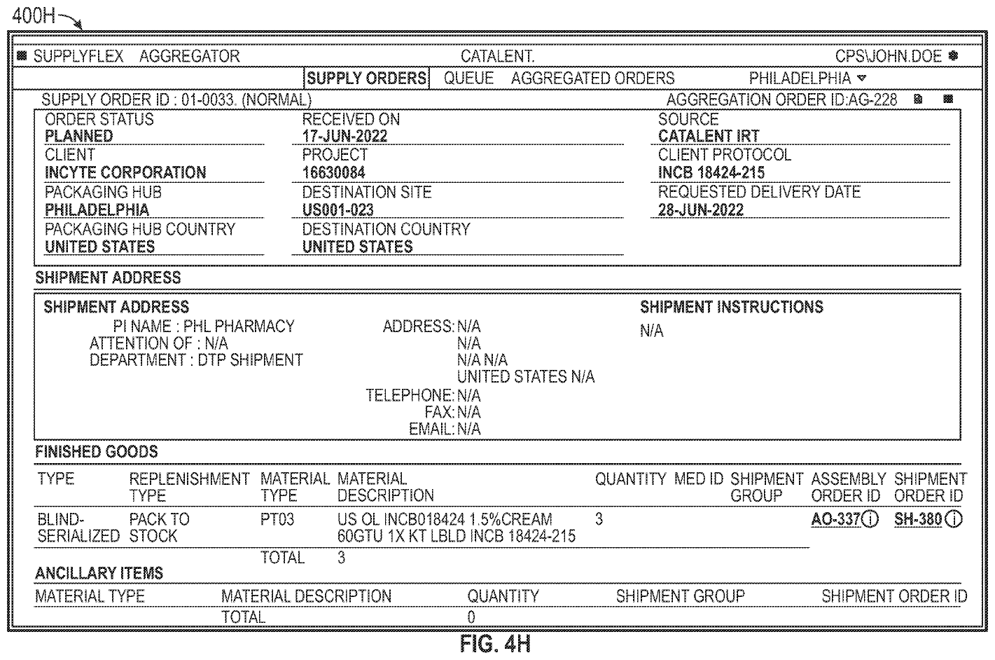

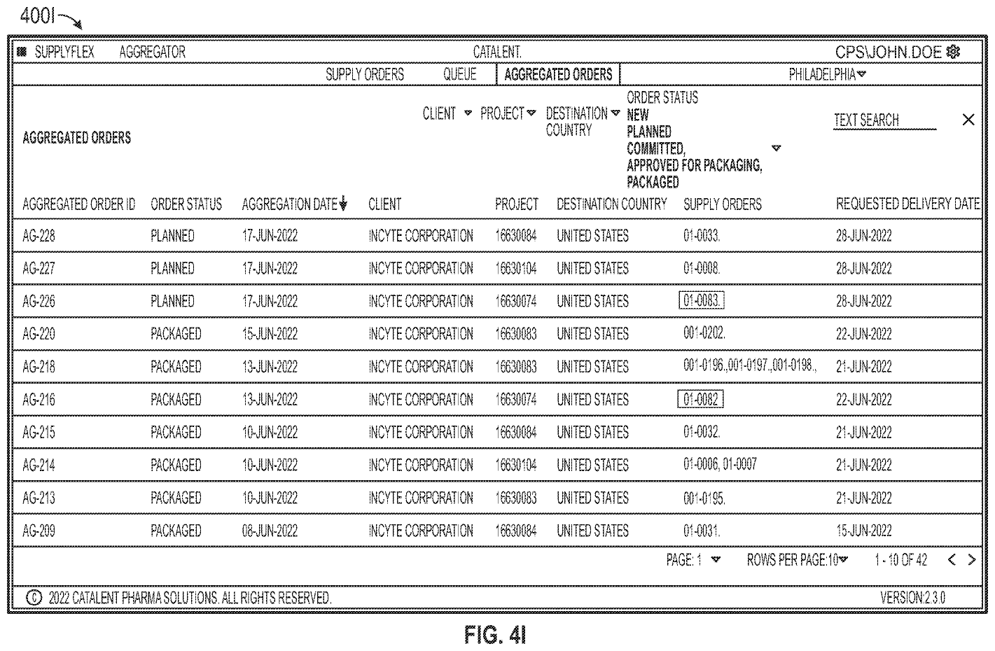

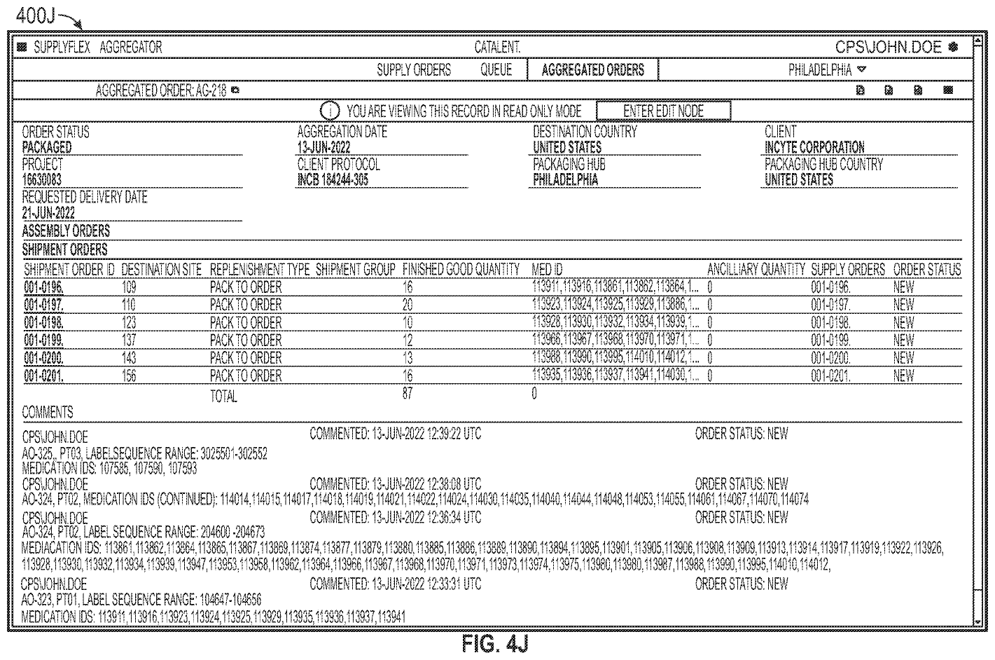

In one or more examples, supply orders that have been assigned to a packaging site may be placed in an aggregator queue (e.g., shown in FIG. 4E). In one or more examples, each packaging site may be associated with a separate queue. In one or more examples, an operator may manually input into the system which supply orders should be aggregated from the queue. In one or more examples, the system may automatically determine which supply orders should be aggregated from the queue. In one or more examples, a user associated with the system may determine which supply orders should be aggregated from the queue. For example, the system can display a preview of the aggregation of expected assembly orders and shipment orders (e.g., shown in FIG. 4F).

In one or more examples, blocks 108 and 110 are associated with a process of aggregating supply orders in accordance with embodiments of the present disclosure. As discussed above, traditionally, each supply order is prepared separately. A supply order that includes multiple packaging types may be prepared in multiple packaging runs. For example, each packaging type may be associated with particular packaging, materials, protocols and requirements, thus products for different packaging types may be produced in separate packaging runs. Packaging the supply orders using these methods can be inefficient, costly, and time consuming when there are multiple supply orders that include multiple packaging types. For example, each packaging run may be associated with a room set-up, a room clean-down, and a separate review and release of the packaged product. Thus, a single supply order may be associated with multiple room set-ups, room clean-downs, and reviews and releases of the packaged product.

At block 108 of FIG. 1, the system can, at the first computer system, associate a first sub-group of the first plurality of supply orders with a first assembly procedure. In one or more examples, the first sub-group of the first plurality of supply orders may correspond to supply orders that correspond to the same packaging type. For example, referring to the supply orders 320A described above with respect to process 300A, the first sub-group of the first plurality of supply orders may correspond to the first packaging type. Referring to supply orders 320A, the first sub-group may correspond to the three products of packaging type 1 from the first supply order (SO1=PTx3), the five products of packaging type 1 from the second supply order (SO2=PTx5), and the two products of packaging type 1 from the third supply order (SO3=PTx2).

At block 110 of FIG. 1, the system can, at the first computer system, associate a second sub-group of the first plurality of supply orders with a second assembly procedure. For example, referring to the supply orders 320A, the second sub-group of the first plurality of supply orders may correspond to the second packaging type. According to this example, the second sub-group may correspond to the three products of packaging type 3 from the first supply order.

To the extent that process 100 includes creating a first and second assembly order, a skilled artisan will understand that the number of assembly orders created is not intended to limit the scope of the present disclosure. More or less assembly orders may be created based on the parameters of a particular clinical trial study. For example, referring to supply orders 320A, the method could further include associating a third sub-group of the first plurality of supply orders with a third assembly procedure.

For example, referring to supply orders 320A, packaging the three supply orders according to this method would yield five separate packaging runs. Two packaging runs associated with the first supply order, e.g., a first packaging run for the first packaging type (SO1=PT1x3) and a second packaging run of a second packaging type (SO1=PT2x3); one packaging run for the second supply order associated with the first packaging type (SO2=PT1x5); and two additional packaging runs for the third supply order, e.g., a first packaging run for the two orders of the first packaging type (SO3=PT1x2) and a second packaging run for two orders of a second packaging type (SO3=PT3x2). Each packaging run may be associated with a separate room set-up, a separate room clean-down, a separate review of the packaged product, and a separate release of the packaged product. The time associated with performing these separate tasks can increase costs, time, and inefficiencies.

FIG. 3B illustrates exemplary aggregated order 300B in accordance with embodiments of the present disclosure. As shown in the figure, the aggregated order 300B can include one or more supply orders 322B, one or more assembly orders 324B, and one or more shipment orders 326B. In one or more examples, FIG. 3B may be associated with steps 108 and 110. As shown in the figure, embodiments of the present disclosure can reduce the number of packaging runs by aggregating orders that are associated with the same packaging type. For example, orders with a same packaging type may be associated with kits including the same type of medication, a same quantity of medication, and similar packaging of the medication. Accordingly, these orders with the same packaging type may be associated with the same protocol (e.g., at least because the packaging type is for the same study within in the same country). Aggregating orders in this manner can improve packaging efficiency by reducing the number of packaging runs and associated overhead (e.g., separate batch record creation, room set-up, room clean-down, review and releases).

The aggregated order 300B may correspond to an exemplary order aggregation for the supply orders 320A. For example supply orders 322B may correspond to the supply orders 320A, where the supply orders may be associated with different site location and one or more packaging types. The site locations may be located in the same country. For example, the first supply order may be associated with three products of a first packaging type (SO1=PT1x3) and three products of a second packaging type (SO1=PT2x3); the second supply order may be associated with five products of the first packaging type (SO2=PT1x5); and the third supply order may be associated with two products of the first packaging type (SO3=PT1x2) and two orders of a second packaging type (SO3=PT3x2).

Assembly orders 324B of FIG. 3B, illustrate the aggregation of the supply orders based on packaging type. For example, as shown in the figure, packaging type 1 is associated with assembly order 1, packaging type 2 is associated with assembly order 2, and packaging type 3 is associated with assembly order 3. Thus, embodiments of the present disclosure can reduce the number of packaging runs associated with these three supply orders from five separate runs associated with the traditional packaging methods to three runs. A skilled artisan will understand that these efficiency gains are even more pronounced as the number of supply orders increases.

While the number of supply orders 322B is the same as the number of assembly orders 324B, a skilled artisan will understand that the number of supply orders may differ from the number of assembly orders based on the packaging types included in the supply orders selected for aggregation.

Shipment orders 326B of FIG. 3B, illustrates how the system can dis-aggregate the assembly orders into shipment orders based on the supply orders. For example, one packaged, the product may be allocated based on the original supply orders 320A. As shown in the figure, shipment order 1 may correspond to supply order 1, such that the quantities of product for various packaging types are the same. For example, shipment order 1 and supply order 1 include three products of a first packaging type and three products of the second packaging type. As shown in the figure, shipment order 2 may correspond to supply order 2 and shipment order 3 may correspond to supply order 3.

FIG. 3C illustrates an exemplary process 300C associated with blocks 108 and 110. At block 302C, the system can create assembly orders based on packaging type. At block 304C, the system can create shipment orders based on the supply order, where there will be one shipment order associated with each site location that has submitted a supply order. Block 302C and block 304C may be associated with aggregated order 300B described above.

In one or more examples, the system may display an aggregation preview based on blocks 302C and 304C. In one or more examples, the system may confirm or receive a confirmation of the aggregation preview. Once confirmed, the system can aggregate the supply orders to produce assembly orders and shipment orders. The system can generate three ID types as part of each new aggregated order: a single Aggregation Order ID assigned to the aggregation, an Assembly Order ID for each assembly orders grouped by packaging type, and a Shipment Order ID for each shipment orders grouped by site and shipment group.

Once the assembly order and shipment orders are created, the system may change an aggregator orders status to new at Block 306C. This status change may indicate to the system that the assembly orders are ready for review prior to sending to a packaging site. For example, the system can confirm the availability of packaging materials (e.g., medication, bottles, blister packs, labels, etc.) at the packaging site. At block 308C, the system can enter labeled lot numbers and expiry dates. In one or more examples, the labeled lot numbers may be received from an external system via an API.

At block 310C, the system can confirm or change the labeled lot numbers and expiry dates for the aggregated orders. In one or more examples, after lot numbers and expiry dates are assigned the system can then mark the aggregated order as planned.

At block 312C, the system can determine whether the labeled lot numbers and expiry dates are approved. If the labeled lot numbers and expiry dates are not approved, the system may, at block 314C wait for approval or change the assembly order by removing one or more supply orders associated with the assembly order. If the labeled lot numbers and expiry dates are approved, the system may, at block 316C display material quantities for the approved labeled lot numbers from an external, integrated system. At block 318C, the system may check to determine whether the materials for packaging the assembly orders are available. In one or more examples the system can confirm the availability of packaging materials (e.g., medication, bottles, blister packs, labels, etc.) at the packaging site. The system can then mark the aggregated order as planned at block 320C.

FIG. 3D illustrates an exemplary process 300D performed by the first computer system and associated with creating the aggregated orders (e.g., aggregator system). Process 300D is a continuation of process 300C. Once the aggregated order status is sent to planned, the system can initially assign the lot identifiers and an expiry date at block 304D and save the order at block 306D. In one or more examples, the system can send a notification to a second computer system associated with the packaging site that a newly created aggregated order has been assigned to the packaging site. In such examples, one or more users associated with the second computer system at the packaging site may review the aggregated order and verify availability of packaging materials at the packaging site, plan packaging runs and assign each assembly order to packaging against the initially assigned lot identifiers and expiry dates. In one or more examples, the lot identifiers may be updated based on client preferences, e.g., the client can specify preferred lot identifiers. Once all the assembly orders are assigned, the aggregated order is marked as committed at step 308D.

In one or more examples, the committed aggregated order may initiate an external process to create work orders and executable batch record which is then used by quality assurance operators users to review each assembly order and mark as approved for packaging at block 314D. In one or more examples, the quality assurance operators may ensure that the lot identifiers correspond to the client specified lot identifiers.

Once the assembly orders are approved at block 310D, the aggregated order is marked as approved for packaging.

In one or more examples the quality assurance operators may approve or reject each assembly order and perform a lot release at block 312D. As a part of the lot release, the system may confirm medication identifiers, usage sequences printed for each serialized assembly orders and shipment orders from external systems. After all assembly orders have been packaged/rejected, the system may receive an indication as to whether an aggregated order was packaged or rejected. In one or more examples, a rejected aggregated order may be processed via a manual process separate from the system.

At block 112 of FIG. 1, the system can, at the second computer system, receive the first assembly order and the second assembly order. In one or more examples, the second computer system may be associated with a packaging facility for packaging the assembly orders. In this manner, the first plurality of supply orders that are associated with the same clinical trial and site locations in the same country may be packaged at the same packaging facility.

At block 114 of FIG. 1, the system can, at the second computer system, assign, randomly, the plurality of kit identifiers to the first assembly order and the second assembly order, thereby creating a plurality of blinded kits. In one or more embodiments, randomly assigning the plurality of kit identifiers may comprise assigning a selected range of pre-randomized kit identifiers. In such embodiments, numbers corresponding to the pre-randomized kit identifiers may be selected without knowledge of the medication type represented by the kit identifier. The assembly orders may be packaged based on this randomization. In one or more examples, while the second computer system associated with the packaging site has access to the randomization list to determine which kits are associated with an active medication or a placebo, the first computer system may not have access to this randomization list. In this manner, the system can confidentially package the original supply order and attenuate the randomization information from the first computer system (e.g., associated with aggregation of the supply orders), thereby reducing the likelihood that a study may become unblinded.

In one or more examples, the kit identifiers may be designated by a customer, e.g., an individual associated with a clinical trial study. For example, the system, at the first computer system, can receive an input from a customer associated with first clinical trial study, where the input includes updated kit identifiers for one or more of the first plurality of supply orders. The system can then update the plurality of kit identifiers associated with the first plurality of supply orders. This updating of the kit identifiers may be completed before assigning the plurality of kit identifiers to the first assembly order and the second assembly order at block 114.

FIG. 3E illustrates an exemplary process 300E associated with block 114. The blocks associated with process 300E may occur at the second computer system associated with the packaging site. At block 302E, the second computer system may randomize kit identifiers based on a packaging type, e.g., by associating the kit identifiers with an active packaging type or a control (e.g., placebo) packaging type. In one or more examples, the active packaging type may be associated with a first packaging type and the control packaging type may be associated with a second, different packaging type. In one or more examples, the kit identifiers may be obtained from the first computer system. In one or more examples, the randomization list associating the kit identifiers to the active or placebo packaging types may be maintained on the second computer system and not shared with the first computer system. In one or more examples, the second computer system may be external to the first computer system.

At block 304E, the system can fulfill the scan assembly order. In one or more examples, the scan assembly order may be performed by an external scan assembly system. The scan assembly system may be configured to verify selected kit IDs and provide control and traceability of the kits movements. In some examples, the scan assembly system may be in communication with the first computer system (e.g., aggregator system). At block 306E, the system can create a master packout. In one or more examples, the data associated with the assembly orders in the first computer system is communicated to the scan assembly system to create the master packout. In one or more examples, at block 308E, the system can associate the packaging jobs (e.g., corresponding to the assembly orders) to a master packout job. For example, the scan assembly system may receive reference labels and the individual kits IDs from the first computer system and the scan assembly system can manage the packing of the shipments ensuring kits IDs are matched to the correct shipment. In one or more examples, at step 310E, the system can create child packout jobs. The child packout jobs may refer to the packaging of each medication type, e.g., as described above one medication may be present in the packaging room at a time. For the child packout jobs, the system may determine at step 312E whether the supply order is quantity driven of kit driven. If the supply order is quantity driven, then the system may determine a quantity of material types from the assembly job at block 314E. If the supply order is kit driven, then the system may obtain aggregated order details, including material identifiers from the first computer system. At block 318E, the system can fulfill the packout job.

At block 116 of FIG. 1, the system can, at the first computer system, confidentially producing shipment orders for the first plurality of clinical trials based on the plurality of blinded kits. For example, the system disaggregate the assembly orders into shipment orders 326B based on the received supply orders 322B as described above with respect to aggregated order 300B. For example, each shipment order may be associated with a single site location and include kits packaged based on one or more assembly orders. As discussed previously, because the randomization information is maintained at the second computer system, the shipment orders may be produced confidentially, e.g., such that one would not be able to unblind the study. In this manner, in addition to improved packaging efficiencies provided by aggregating supply orders, embodiment of the present disclosure provide improved security in preparing supply orders for clinical trial studies.

In one or more examples, the system may receive an indication that the packout job has been fulfilled and that the assembly orders have been packaged. In one or more examples, the second system may communicate to the first system that the assembly orders have been packaged. In one or more examples, the system may first perform a quality assurance check on the packaged assembly orders. If an assembly order does not pass a quality assurance check, the assembly order may be flagged for a manual review and/or shipment process. A lot release may be performed following the quality assurance check. The lot release may confirm material identifiers, usage sequences printed for each assembly order and usage sequences for shipment orders from external systems or applications.

Embodiments of the present disclosure further provide flexibility to implement both pack to order and pack to stock shipment types. As used herein, pack to order may refer to supply orders that are received by the system and should subsequently be shipped to a designated site location associated with a clinical trial. As used herein, pack to stock may refer to supply orders that are received by the system and should be subsequently stored at a facility (e.g., a packaging facility or designated warehouse) to be shipped to a site location at a later date. In one or more examples, the site location associated with the pack to stock shipment type may not be known when the supply order is placed.

In one or more examples, the system can, at the first computer system, determine a shipment type for a first supply order of the first plurality of supply orders. If a supply order is associated with a pack to order shipment type, the first system can produce the shipment order as described above. For example, the first shipment order can be confidentially produced based on the plurality of blinded kits. The packaged kits associated with the first shipment order may then be shipped to the appropriate clinical trial site location.

If a supply order is associated with a pack to stock shipment type, the first computer system may produce instructions to store the kits produced based on the supply order. The kits may be stored at the packaging facility or warehouse facility. At a later time, the first computer system may receive a request from a first clinical trial site requesting materials associated with the supply order. In such examples, the system can produce a shipment order for the supply order based on the request from the clinical trial site location. In this manner, the customer may keep an amount of material packaged as a reserve. This may provide the customer with more flexibility to reach additional participants in clinical trial studies by having access to pre-packaged materials, without having to go through a new packaging run.

In one or more examples, embodiments of the present disclosure can provide systems and methods for aggregating supply orders associated with a clinical trial site location into assembly orders based on one or more treatment types (e.g., packaging types). The kits produced via the assembly orders may be disaggregated into one or more shipment orders based on the supply orders. In one or more examples, the aggregation process can include at least a planning stage, a post-production stage, and a shipments stage. These stages may be associated with one or more blocks described above with respect to process 100.

In one or more examples, as a part of the planning stage, the system may receive a plurality of supply orders from an external system or application, e.g., when the external system finds a site demand or when the system receives a direct request from site location associated with a client. In one or more examples, the system may automatically assign a packaging location to these supply orders based on destination location. In one or more examples, the packaging location may be based on whether the site locations are located in the same country. In one or more examples, the packaging location may be based on whether the site locations are located in the same region. In one or more examples, the packaging location may be pre-configured such that when a request from a particular site location is received, the packaging location may be automatically assigned.

In one or more examples, supply orders that have been assigned to a packaging site may be placed in an aggregator queue (e.g., shown in FIG. 4E). In one or more examples, each packaging site may be associated with a separate queue. In one or more examples, the system may automatically determine which supply orders should be aggregated from the queue. In one or more examples, a user associated with the system may determine which supply orders should be aggregated from the queue. For example, the system can display a preview of the aggregation of expected assembly orders and shipment orders (e.g., shown in FIG. 4F).

The system may confirm or receive a confirmation of the aggregation preview. Once confirmed, the system can aggregate the supply orders to produce assembly orders and shipment orders. The system can generate three ID types as part of each new aggregated order: a single Aggregation Order ID assigned to the aggregation, an Assembly Order ID for each assembly orders grouped by packaging type, and a Shipment Order ID for each shipment orders grouped by site and shipment group.

In one or more examples the system can confirm the availability of packaging materials (e.g., medication, bottles, blister packs, labels, etc.) at the packaging site. The system can further initially assign the lot identifiers and an expiry date. The system can then mark the aggregated order as planned. Once planned, the system can send a notification to a second computer system associated with the packaging site that a newly created aggregated order has been assigned to the packaging site. In such examples, one or more users associated with the second computer system at the packaging site may review the aggregated order and verify availability of packaging materials at the packaging site, plan packaging runs and assign each assembly order to packaging against the initially assigned lot id's and expiry dates. In one or more examples, the lot identifiers may be updated based on client preferences, e.g., the client can specify preferred lot identifiers. Once all the assembly orders are assigned, the aggregated order is marked as committed.

In one or more examples, the committed aggregated order may initiate an external process to create work orders and executable batch record which is then used by quality assurance operators users to review each assembly order and mark as approved for packaging. In one or more examples, the quality assurance operators may ensure that the lot identifiers correspond to the client specified lot identifiers. Once the assembly orders are approved, the aggregated order is marked as approved for packaging. In one or more examples, aggregated orders may include only ancillaries. In such examples, the aggregated order would go directly into post-production without planning activities.

In one or more examples, as a part of the post-production stage, the quality assurance operators may approve or reject each assembly order and perform a lot release. As a part of the lot release, the system may confirm medication identifiers, usage sequences printed for each serialized assembly orders and shipment orders from external systems. After all assembly orders have been packaged/rejected, the system may receive an indication as to whether an aggregated order was packaged or rejected. In one or more examples, a rejected aggregated order may be processed via a manual process separate from the system.

In one or more examples, the system may receive an indication of whether ancillary lots are a part of the aggregated order. In one or more examples, ancillary lots may refer to non-manufactured purchased items to be included within an order (e.g., syringes, alcohol based wipes). These ancillary items may be considered generic and non-unique.

In one or more examples, once the assembly orders are packaged and ancillary information has been entered, the shipment orders may be ready for pre-distribution. In one or more examples, as a part of the shipment stage, the system can mark the aggregated order as approved for shipment which can generate a shipment plan for each of the shipment orders with a shipment type of Pack to Order (PTO) within the aggregation. A shipment plan will not be created for Pack to Stock (PTS) shipment types. PTS orders will be packaged and will remain in at the packaging facility or warehouse.

In one or more examples, the system may receive a shipment order message, at the second computer system associated with the packaging site, from an external system. The system may initiate the shipment process and update the status of the shipment orders associated with an aggregated order to a sent status. If an error occurs with the shipment process, the system may flag a manual review of the shipment prior to notifying the external system. The system may receive verification from one or more external systems that the shipment of the product has been shipped or delivered and update the status of the aggregated order accordingly.

FIGS. 4A-4J illustrate exemplary user interfaces in accordance with embodiments of the present disclosure.

FIG. 4A illustrates an exemplary user interface 400A corresponding to a log in screen for the first computer system. In one or more examples, a user associated with the first computer system may be prompted to log in to the first computer system via a user interface 400A. As shown in the figure, the user interface 400A may include one or more user affordances to receive from a user, a username and a password. The user interface may further include a user affordance (e.g., log in button) for a user to log in to the first computer system upon providing a user name and password.

FIG. 4B illustrates an exemplary user interface 400B corresponding to a landing page for the first computer system. For example, exemplary user interface 400B may be presented to a user upon a successful login. User interface 400B may include a plurality of user affordances for navigating to different sections of the first computer system. For example user interface 400B includes an aggregator user affordances, an admin user affordance and a scan assembly user affordance. User interface 400B may further include a summary of previous log in attempts by the user. This may be provided as a security measure for a user to ensure that there has not been recent unauthorized access of their account.

FIG. 4C illustrates an exemplary user interface 400C corresponding to an administrative page for the first computer system. As shown in the figure, the administrative page may include a navigation bar that allows the user to navigate to one or more user interfaces associated with users, external systems and programs, projects, and an audit trail.

FIG. 4D illustrates an exemplary user interface 400D corresponding to an exemplary aggregator page for the first computer system. As shown in the figure, the aggregator page 400D may be associated with a queue section. The queue section can be adapted to display one or more queued supply orders. For example, the queued supply orders can be displayed based on a selected criteria including a client, a project (e.g., clinical trial study), a destination country, an order type (e.g., initial, normal, emergency), and/or a text search. As used herein an initial order may refer to a first order for a site. As used herein a normal order may refer to a standard order for a site, which may be executed with a standard lead time. As used herein, an emergency order may correspond to an order which needs to be expedited and completed more quickly than the standard lead time. Each of these items may be selected based on a drop down menu, or in the case of the text search, the system may receive the text via a user affordance. Queued supply orders based on the selected information may be displayed on aggregator page 400D.

In one or more examples, the user interface may be adapted to display information relating to the supply order identifier, a material type (e.g., medication and dosage, packaging materials, labeling materials, etc.), when the supply order was received, a destination site (e.g., where the supply order should be shipped), and a requested delivery date, for the queued supply orders. In or more examples, the information displayed in the list of the queued orders may be based on the selected criteria. For example, if a destination country is selected, the displayed information may omit the destination country in the list of the queued orders (e.g., a column corresponding to a destination country may be omitted). This may reduce clutter and enhance readability of the user interface by eliminating redundant information from being presented.

FIG. 4E illustrates an exemplary user interface 400E corresponding to an exemplary aggregator page for the first computer system. As shown in the figure, the aggregator page 400E may be associated with a queue section where the system has received information related to a selected client, project (e.g., clinical trial study), destination country, and order type (e.g., initial, normal, emergency). As shown in the figure, the system can display a plurality of queued orders based on the received information. As shown in the figure, multiple supply orders may be associated with the same destination site and/or material type.

For example, supply order ID 01-0079 placed on Jun. 4, 2022, may be associated with material type 3 and correspond to a requested quantity of 32 to be shipped to destination site 16; supply order ID 01-0074 placed on Jun. 4, 2022, may be associated with material type 3 and correspond to a requested quantity of 13 to be shipped to destination site 6; supply order ID 01-0073 placed on Jun. 4, 2022, may be associated with material type 3 and correspond to a requested quantity of 19 to be shipped to destination site 5; supply order ID 01-0072 placed on Jun. 4, 2022, may be associated with material type 3 and correspond to a requested quantity of 9 to be shipped to destination site 4; supply order ID 01-0072 placed on May 26, 2022, may be associated with material type 3 and correspond to a requested quantity of 25 to be shipped to destination site 6; supply order ID 01-0066 placed on May 25, 2022, may be associated with the same material type 3 and correspond to a requested quantity of 13 to be shipped to destination site 5; supply order ID 01-0068 placed on May 25, 2022, may be associated with the same material type 3 and correspond to a requested quantity of 9 to be shipped to destination site 4; and supply order ID 01-0066 placed on May 24, 2022, may be associated with the same material type 3 and correspond to a requested quantity of 13 to be shipped to destination site 4. According to embodiments of this disclosure because these supply orders enumerated above correspond to the same material type (e.g., packaging type), the same client, the same clinical trial study, and the same destination country, the system may aggregate these orders together for packaging. In contrast, previous systems may have produced separate packaging runs for each of the orders.

While each of the client, project, destination country, and order type fields are selected in user interface 400E, in one or more examples, more or less fields may be utilized to display a list of queued supply orders. For example, the system can display queued supply orders based on the client, and project.

FIG. 4F illustrates an exemplary user interface 400F corresponding to an exemplary aggregator page for the first computer system. As shown in the figure, the aggregator page 400F may be associated with an aggregation preview. The aggregation preview may include an assembly order section that displays a list corresponding to the different assembly orders and a shipment orders section that displays a list corresponding to the different shipment orders. In one or more examples, the system may receive a selection of one or more supply orders listed in the queue shown in user interface 400E. For example, user interface 400F may be based on a selection of supply order IDs 01-0081 and 01-0079 shown in user interface 400E. As shown in the figure, the user interface may provide user affordances to confirm or cancel the displayed aggregation preview. Confirming the aggregation preview may remove the corresponding supply orders from the queue.

Referring to user interface 400F, the assembly order section can include information regarding the type of study (e.g., blinded or open), a material type (e.g., packaging type), a material description (e.g., the medication and dosage), and kit identifiers. As shown in the figure, while the assembly order associated with material type 3 is indicated as a blinded study, unblinding information may not be displayed or accessible to the user via user interface 400F.

The shipment order section can include information regarding the destination site, the replenishment type (e.g., pack to order or pack to ship), a shipment group, a finished good quantity, and a kit identifier. In one or more examples, the shipment order information may be based on the supply order information.

FIG. 4G illustrates an exemplary user interface 400G corresponding to an exemplary aggregator page for the first computer system. The user interface 400G may be associated with a supply orders page. As shown in the figure, user interface 400G may display a list of supply orders. The list of supply orders may be displayed based on to a selected client, project (e.g., clinical trial study), destination country, order type (e.g., initial, normal, emergency), order status (e.g., queued, new, planned) and or a text input received from a user. The supply order list may be displayed based on one or more of these parameters. For each supply order included in the list, the user interface 400G can display, but is not limited to, a supply order identifier, an order status, an aggregated order identifier, a date the supply order was received, a name of the client, a project (e.g., clinical trial study), a source (e.g., whether the order was received via an application or direct customer communication), a requested delivery date, a destination site, and a destination country. As shown in user interface 400G, the supply order list corresponds to a selection of an order type and order status. In one or more examples, the information displayed in the list of the queued orders may be based on the selected criteria. For example, if a destination country is not selected, the displayed information may include the destination country in the list of the queued orders (e.g., a column corresponding to a destination country may be omitted), as shown in user interface 400G. This may reduce clutter and enhance the readability of the user interface.

FIG. 4H illustrates an exemplary user interface 400H corresponding to an exemplary aggregator page for the first computer system. The user interface 400H may correspond to a supply order detail page associated with a selection of a particular supply order (e.g. supply order identifier: 01-0033) from the supply order page (e.g., user interface 400G). As shown in user interface 400H, the supply order detail page can display information associated with, but is not limited to, the supply order including general information including an order status, a date the supply order was received, a name of the client, a project (e.g., clinical trial study), a source (e.g., whether the order was received via an application or direct customer communication), a client protocol identifier, a packaging hub location including country and city (e.g., where the supply order will be packaged), a requested delivery date, a destination site, and a destination country. The user interface 400H may also display shipment details including shipment address and shipment instructions. The user interface 400H may also display finished goods information that includes, but is not limited to, a study type (e.g., blinded or open), a replenishment type (e.g., pack to order and pack to stock), a material type, a material description, a quantity, an assembly order ID and a shipment order ID. The user interface 400H may also include information regarding any ancillary items related to the supply order. A skilled artisan will understand that these user interfaces are exemplary and more or less information may be included without departing from the scope of this disclosure.

FIG. 4I illustrates an exemplary user interface 400I corresponding to an exemplary aggregated orders page for the first computer system. The user interface 400I may display a list of aggregated orders based on one or more selected criteria. As shown in the figure, the one or more selected criteria may include a client, a project, a destination country, an order status, or a text search. The aggregated orders may correspond to one or more of the aggregated orders described above with respect to blocks 108 and 110, and FIGS. 3B and 3C. The information displayed for each of the aggregated orders may include, but is not limited to, an aggregated order identifier, an order status, an aggregation date, a client, a project (e.g., clinical trial study), a destination country, one or more supply orders that comprise the aggregated order, and a requested delivery date. In or more examples, the information displayed in the list of the queued orders may be based on the selected criteria. For example, if a destination country is not selected, the displayed information may include the destination country in the list of the queued orders (e.g., a column corresponding to a destination country may be omitted), as shown in user interface 400I.

FIG. 4J illustrates an exemplary user interface 400I corresponding to an exemplary aggregated orders page for the first computer system. The user interface 400I corresponds to a details page associated with a particular aggregated order, e.g., aggregated order AG-218. As shown in the figure, the aggregated order details page may include general information related to the aggregated order, including, but not limited to, a client, an order status (e.g., packaged, planned, queued, new, shipped), an aggregation date, a project (e.g., clinical trial study), a requested delivery date, a client protocol number, a destination country, a packaging hub (including city and country). The user interface 400I may further include details regarding the shipment orders. In one or more examples, the shipment orders can correspond to the shipment orders described above with respect to blocks 116 and 326B. For example, the shipment orders can correspond to the de-aggregated assembly orders. User interface 400I may display information including, but not limited to, the shipment order identifier, a destination site, a replenishment type, a shipment group, a finished good quantity, a kit identifier, an ancillary quantity, a supply order number, and an order status. The user interface 400I may further include one or more comments. In addition to the comment itself, the one or more comments may identify the user making the comment, a date corresponding to when the comment was posted.

A skilled artisan will understand that these user interfaces are exemplary and more or less information may be included without departing from the scope of this disclosure. Additionally, different display configurations may be used without departing from the scope of this disclosure.

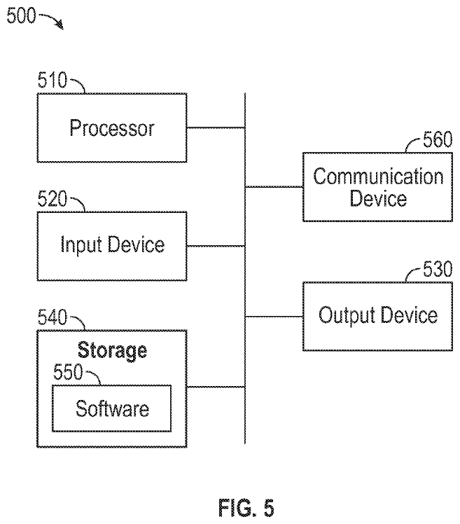

The operations described above with reference to the above-described figures are optionally implemented by components depicted in FIG. 5. It would be clear to a person having ordinary skill in the art how other processes are implemented based on the components depicted in FIG. 5.

FIG. 5 illustrates an example of a computing device in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more of processor 510, input device 520, output device 530, storage 540, and communication device 560. Input device 520 and output device 530 can generally correspond to those described above, and can be either connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage, such as an electrical, magnetic or optical memory including a RAM, cache, hard drive, or removable storage disk. Communication device 560 can include any suitable device capable of transmitting and receiving signals over a network, such as a network interface chip or device. The components of the computer can be connected in any suitable manner, such as via a physical bus or wirelessly.

Software 550, which can be stored in storage 540 and executed by processor 510, can include, for example, the programming that embodies the functionality of the present disclosure (e.g., as embodied in the devices as described above).

Software 550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 540, that can contain or store programming for use by or in connection with an instruction execution system, apparatus, or device.

Software 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can implement any operating system suitable for operating on the network. Software 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Embodiments of the present disclosure provide methods for confidentially producing shipment orders for clinical trials. In one or more examples, the method comprises at a first computer system comprising one or more processors and one or more memories: receiving a plurality of supply orders associated with one or more clinical trials, determining for each of the plurality of supply orders: a clinical trial study, a site location, and a treatment type, assigning a first plurality of supply orders associated with a first clinical trial study to a packaging site based on the site location. In one or more examples, the method further comprises associating a first sub-group of the first plurality of supply orders with a first assembly procedure based on the treatment type to create a first assembly order, and associating a second sub-group of the first plurality of supply orders with a second assembly procedure based on the treatment type to create a second assembly order. In one or more examples, the method further comprises at a second computer system comprising a second one or more processors and a second one or more memories: receiving the first assembly order and the second assembly order; and assigning, randomly, a plurality of kit identifiers to the first assembly order and the second assembly order, thereby creating a plurality of blinded kits. In one or more examples, the method further comprises at the first computer system, confidentially producing shipment orders for the first plurality of clinical trials based on the plurality of blinded kits.

In some embodiments, the first treatment type corresponds to an active group and the second treatment type corresponds to a control group. In some embodiments, the method further comprises, at the first computer system: determining a shipment type for the first plurality of supply orders, in accordance with a determination one or more supply orders are associated with a first shipment type, producing shipment orders for the one or more supply orders associated with the first shipment type, and in accordance with a determination one or more supply orders are associated with a second shipment type, producing instructions to store the one or more supply orders associated with the second shipment type. In some embodiments, the first shipment type corresponds to a pack-to-order request and the second shipment type corresponds to a pack-to-stock request. In some embodiments, the method further comprises receiving a request from a first clinical trial site associated with a first supply order of the one or more supply orders associated with the second shipment type; and producing a shipment order for the first supply order based on the request from the first clinical trial site.

In some embodiments, the determined site locations of the first plurality of supply orders are associated with a same country. In some embodiments, the plurality of supply orders are received via a customer communication or an application programming interface associated with a client.

In some embodiments, the method further comprises, at the first computer system: receiving an input from a client associated with the first clinical trial study corresponding to updated kit identifiers for one or more of the first plurality of supply orders, and updating the plurality of kit identifiers associated with the first plurality of supply orders. In some embodiments, updating the plurality of kit identifiers is performed prior to assigning the plurality of kit identifiers to the first assembly order and the assembly order.

In some embodiments, the method further comprises, at the first computer system receiving a rejection from a user associated with the first computer system for the first assembly order, the second assembly order, or a combination thereof, and flagging a rejected assembly order for manual shipment processes.

In one or more examples, embodiments of the present disclosure include an electronic system comprising one or more processors, a memory, and one or more programs, wherein the one or more programs are stored in the memory and configured to be executed by the one or more processors. In one or more examples, the one or more programs further include instructions for at a first computer system comprising one or more processors and one or more memories: receiving a plurality of supply orders associated with one or more clinical trials, determining for each of the plurality of supply orders: a clinical trial study, a site location, and a treatment type, assigning a first plurality of supply orders associated with a first clinical trial study to a packaging site based on the site location. In one or more examples, the one or more programs further include instructions for associating a first sub-group of the first plurality of supply orders with a first assembly procedure based on the treatment type to create a first assembly order, and associating a second sub-group of the first plurality of supply orders with a second assembly procedure based on the treatment type to create a second assembly order. In one or more examples, the one or more programs include instructions for at a second computer system comprising a second one or more processors and a second one or more memories: receiving the first assembly order and the second assembly order, and assigning, randomly, a plurality of kit identifiers to the first assembly order and the second assembly order, thereby creating a plurality of blinded kits. In one or more examples, the one or more programs include instructions for at the first computer system, confidentially producing shipment orders for the first plurality of clinical trials based on the plurality of blinded kits.

In some embodiments, the first treatment type corresponds to an active group and the second treatment type corresponds to a control group. In some embodiments, the one or more programs further include instructions for, at the first computer system, determining a shipment type for the first plurality of supply orders, in accordance with a determination one or more supply orders are associated with a first shipment type, producing shipment orders for the one or more supply orders associated with the first shipment type, and in accordance with a determination one or more supply orders are associated with a second shipment type, producing instructions to store the one or more supply orders associated with the second shipment type. In some embodiments, the first shipment type corresponds to a pack-to-order request and the second shipment type corresponds to a pack-to-stock request. In some embodiments, the one or more programs further include instructions for, at the first computer system, receiving a request from a first clinical trial site associated with a first supply order of the one or more supply orders associated with the second shipment type; and producing a shipment order for the first supply order based on the request from the first clinical trial site.

In some embodiments, the determined site locations of the first plurality of supply orders are associated with a same country. In some embodiments, the plurality of supply orders are received via a customer communication or an application programming interface associated with a client.

In some embodiments, the one or more programs further include instructions for, at the first computer system, receiving an input from a client associated with the first clinical trial study corresponding to updated kit identifiers for one or more of the first plurality of supply orders, and updating the plurality of kit identifiers associated with the first plurality of supply orders. In some embodiments, updating the plurality of kit identifiers is performed prior to assigning the plurality of kit identifiers to the first assembly order and the assembly order.

In some embodiments, the one or more programs further include instructions for, at the first computer system, receiving a rejection from a user associated with the first computer system for the first assembly order, the second assembly order, or a combination thereof, and flagging a rejected assembly order for manual shipment processes.

In one or more examples, embodiments of the present disclosure further include a non-transitory computer-readable storage medium storing one or more programs, the one or more programs comprising instructions, which when executed by one or more processors of one or more electronic devices having a display, cause the one or more electronic devices to: at a first computer system comprising one or more processors and one or more memories, receive a plurality of supply orders associated with one or more clinical trials, determine for each of the plurality of supply orders: a clinical trial study, a site location, and a treatment type, assign a first plurality of supply orders associated with a first clinical trial study to a packaging site based on the site location. In one or more examples, the one or more electronic devices are further caused to: associate a first sub-group of the first plurality of supply orders with a first assembly procedure based on the treatment type to create a first assembly order, and associate a second sub-group of the first plurality of supply orders with a second assembly procedure based on the treatment type to create a second assembly order. In one or more examples, the one or more electronic devices are further caused to: at a second computer system comprising a second one or more processors and a second one or more memories, receive the first assembly order and the second assembly order, and assign, randomly, a plurality of kit identifiers to the first assembly order and the second assembly order, thereby creating a plurality of blinded kits. In one or more examples, the one or more electronic devices are further caused to at the first computer system, confidentially produce shipment orders for the first plurality of clinical trials based on the plurality of blinded kits.

In some embodiments, the first treatment type corresponds to an active group and the second treatment type corresponds to a control group. In some embodiments, the one or more electronic devices are further caused to, at the first computer system, determine a shipment type for the first plurality of supply orders, in accordance with a determination one or more supply orders are associated with a first shipment type, produce shipment orders for the one or more supply orders associated with the first shipment type, and in accordance with a determination one or more supply orders are associated with a second shipment type, produce instructions to store the one or more supply orders associated with the second shipment type. In some embodiments, the first shipment type corresponds to a pack-to-order request and the second shipment type corresponds to a pack-to-stock request. In some embodiments, the one or more electronic devices are further caused to, at the first computer system, receive a request from a first clinical trial site associated with a first supply order of the one or more supply orders associated with the second shipment type, and produce a shipment order for the first supply order associated with the second shipment type based on the request from the first clinical trial site.

In some embodiments, the determined site locations of the first plurality of supply orders are associated with a same country. In some embodiments, the plurality of supply orders are received via a customer communication or an application programming interface associated with a client.

In some embodiments, the one or more electronic devices are further caused to, at the first computer system, receive an input from a client associated with the first clinical trial study corresponding to updated kit identifiers for one or more of the first plurality of supply orders, and update the plurality of kit identifiers associated with the first plurality of supply orders. In some embodiments, updating the plurality of kit identifiers is performed prior to assigning the plurality of kit identifiers to the first assembly order and the assembly order.

In some embodiments, the one or more electronic devices are further caused to, at the first computer system, receive a rejection from a user associated with the first computer system for the first assembly order, the second assembly order, or a combination thereof, and flag a rejected assembly order for manual shipment processes.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A method for confidentially producing shipment orders for clinical trials, the method comprising:
at a first computer system comprising one or more processors and one or more memories:
receiving a plurality of supply orders associated with one or more clinical trials;
determining for each of the plurality of supply orders: a clinical trial study, a site location, and a treatment type;
assigning a first plurality of supply orders associated with a first clinical trial study to a packaging site based on the site location;
associating a first sub-group of the first plurality of supply orders with a first assembly procedure based on the treatment type to create a first assembly order; and
associating a second sub-group of the first plurality of supply orders with a second assembly procedure based on the treatment type to create a second assembly order;
at a second computer system comprising a second one or more processors and a second one or more memories:
receiving the first assembly order and the second assembly order; and
assigning, randomly, a plurality of kit identifiers to the first assembly order and the second assembly order, thereby creating a plurality of blinded kits; and
at the first computer system, confidentially producing shipment orders for the first plurality of clinical trials based on the plurality of blinded kits.

2. The method of claim 1, wherein the first treatment type corresponds to an active group and the second treatment type corresponds to a control group.

3. The method of claim 1, further comprising, at the first computer system:
determining a shipment type for the first plurality of supply orders;
in accordance with a determination one or more supply orders are associated with a first shipment type, producing shipment orders for the one or more supply orders associated with the first shipment type; and
in accordance with a determination one or more supply orders are associated with a second shipment type, producing instructions to store the one or more supply orders associated with the second shipment type.

4. The method of claim 3, wherein the first shipment type corresponds to a pack-to-order request and the second shipment type corresponds to a pack-to-stock request.

5. The method of claim 3, further comprising:
receiving a request from a first clinical trial site associated with a first supply order of the one or more supply orders associated with the second shipment type; and
producing a shipment order for the first supply order based on the request from the first clinical trial site.

6. The method of claim 1, wherein the determined site locations of the first plurality of supply orders are associated with a same country.

7. The method of claim 1, wherein the plurality of supply orders are received via a customer communication or an application programming interface associated with a client.

8. The method of claim 1, further comprising, at the first computer system:
receiving an input from a client associated with the first clinical trial study corresponding to updated kit identifiers for one or more of the first plurality of supply orders; and
updating the plurality of kit identifiers associated with the first plurality of supply orders.

9. The method of claim 8, wherein updating the plurality of kit identifiers is performed prior to assigning the plurality of kit identifiers to the first assembly order and the assembly order.

10. The method of claim 1, further comprising, at the first computer system:
receiving a rejection from a user associated with the first computer system for the first assembly order, the second assembly order, or a combination thereof;
flagging a rejected assembly order for manual shipment processes.

11. An electronic system, comprising:
one or more processors;
a memory; and
one or more programs, wherein the one or more programs are stored in the memory and configured to be executed by the one or more processors, the one or more programs including instructions for:
at a first computer system comprising one or more processors and one or more memories:
receiving a plurality of supply orders associated with one or more clinical trials;
determining for each of the plurality of supply orders: a clinical trial study, a site location, and a treatment type;
assigning a first plurality of supply orders associated with a first clinical trial study to a packaging site based on the site location;
associating a first sub-group of the first plurality of supply orders with a first assembly procedure based on the treatment type to create a first assembly order; and
associating a second sub-group of the first plurality of supply orders with a second assembly procedure based on the treatment type to create a second assembly order;
at a second computer system comprising a second one or more processors and a second one or more memories:
receiving the first assembly order and the second assembly order; and
assigning, randomly, a plurality of kit identifiers to the first assembly order and the second assembly order, thereby creating a plurality of blinded kits; and
at the first computer system, confidentially producing shipment orders for the first plurality of clinical trials based on the plurality of blinded kits.

12. (canceled)

13. (canceled)

14. (canceled)

15. (canceled)

16. (canceled)

17. (canceled)

18. (canceled)

19. (canceled)

20. (canceled)

21. A non-transitory computer-readable storage medium storing one or more programs, the one or more programs comprising instructions, which when executed by one or more processors of one or more electronic devices having a display, cause the one or more electronic devices to:
at a first computer system comprising one or more processors and one or more memories:
receive a plurality of supply orders associated with one or more clinical trials;
determine for each of the plurality of supply orders: a clinical trial study, a site location, and a treatment type;
assign a first plurality of supply orders associated with a first clinical trial study to a packaging site based on the site location;
associate a first sub-group of the first plurality of supply orders with a first assembly procedure based on the treatment type to create a first assembly order; and
associate a second sub-group of the first plurality of supply orders with a second assembly procedure based on the treatment type to create a second assembly order;
at a second computer system comprising a second one or more processors and a second one or more memories:
receive the first assembly order and the second assembly order; and
assign, randomly, a plurality of kit identifiers to the first assembly order and the second assembly order, thereby creating a plurality of blinded kits; and
at the first computer system, confidentially produce shipment orders for the first plurality of clinical trials based on the plurality of blinded kits.

22. (canceled)

23. (canceled)

24. (canceled)

25. (canceled)

26. (canceled)

27. (canceled)

28. (canceled)

29. (canceled)

30. (canceled)
